(19) United States
(12) Patent Application Publication
(10) Pub. No.: US 2019/0261960 A1
(43) Pub. Date: Aug. 29, 2019

(54) IMAGE PROCESSING AND ANALYZING SYSTEM FOR OVULATION DETECTION AND METHOD FOR CONTROLLING SAME

(71) Applicant: ManiGene Co., Ltd., Seoul (KR)

(72) Inventor: Jae Min LEE, Seoul (KR)

(73) Assignee: ManiGene Co., Ltd., Seoul (KR)

(21) Appl. No.: 16/346,018

(22) PCT Filed: Nov. 23, 2016
(86) PCT No.: PCT/KR2016/013570

§ 371 Date: Apr. 29, 2019

(30) Foreign Application Priority Data
Oct. 28, 2016 (KR) 10-2016-0142159
Nov. 23, 2016 (KR) 10-2016-0156481
Nov. 23, 2016 (KR) 10-2016-0156482
Nov. 23, 2016 (KR) 10-2016-0156483

(51) Int. Cl. A61B 10/00; G06K 9/00; G06T 7/62; A61B 5/00

(52) U.S. Cl. CPC A61B 10/0012; G06K 9/00134; G06K 9/0014; A61B 2010/0025; G06T 7/62; A61B 10/0051; A61B 5/7264; G06K 9/00147

(57) ABSTRACT

An image processing and analyzing system for ovulation detection includes: an electronic device including a camera portion; a saliva acquisition device including a saliva accommodation portion accommodating saliva of a subject, and a case in which the saliva accommodation portion is located, the case including an entrance, at an end thereof, an entrance through which the saliva accommodation portion enters and exits, and a penetration hole is formed in one surface of the case through which the saliva may be exposed to outside; an ovulation prediction device including a body portion, wherein the electronic device is mounted on one surface and an opening is formed in the surface to allow the camera portion to be positioned in the opening, an insertion unit formed at one side of the body portion to allow the saliva acquisition device to be inserted into a certain depth, and a light source located in the body portion and providing light to the saliva acquisition device; and a controller acquiring a captured image of the saliva from the camera portion, extracting a crystal structure image of hormone included in the saliva by processing the acquired captured image, and determining, based on the extracted crystal structure image, whether the subject is in ovulation.

### TECHNICAL FIELD

The present disclosure relates to an image processing and analyzing system for ovulation detection and a method of controlling the same.

### BACKGROUND ART

Due to social changes, importance of pregnancy plans and contraception plans has gradually increased. Contraception methods mainly include natural contraception based on ovulation cycles of women and artificial contraception using drugs or tools, and as for pregnancy, a method of adjusting dates for sexual intercourses in consideration of ovulation cycles of women and artificial methods of in vitro fertilization and internal fertilization are widely used. However, artificial methods have high possibility of success, whereas a body of a woman or a man may be adversely affected. Therefore, natural methods in consideration of ovulation cycles of women are being recommended.

A method of measuring an ovulation state of a woman is classified into various ways according to parameters for determining whether the woman is in the ovulation state, and above all, a method of measuring an amount of lutein hormone from human body fluid and determining whether the woman is in the ovulation state by using the amount of lutein hormone may be understood as being the most accurate. However, as such method requires specific safety rules, there is inconvenience that tests have to be requested to experts from designated institutions, women tend to avoid the method.

On the contrary, a method of determining whether a woman is in the ovulation state by measuring a basal body temperature of the woman or a method of determining whether a woman is in the ovulation by acquiring mucus in the neck and examining crystals in a process of drying the mucus is very convenient and most easily used by women. However, the crystals in the mucus and the basal body temperature of women may be changed due to external factors such as a disease or environmental changes, and therefore, reliability of determining the ovulation state is greatly decreased.

### DESCRIPTION OF EMBODIMENTS

### Technical Problem

Provided are an image processing and analyzing system, whereby an ovulation may be detected by analyzing an image of saliva, and a method of controlling the image processing and analyzing system.

### Solution to Problem

An embodiment of the present disclosure provides an image processing and analyzing system for ovulation detection including: an electronic device including a camera portion; a saliva acquisition device including a saliva accommodation portion accommodating saliva of a subject, and a case in which the saliva accommodation portion is located, the case including an entrance, at an end thereof, through which the saliva accommodation portion enters and exits, and a penetration hole is formed in one surface of the case through which the saliva is exposed to outside; an ovulation prediction device including a body portion, wherein the electronic device is mounted on one surface and an opening is formed in the surface to allow the camera portion to be positioned in the opening, an insertion unit formed at one side of the body portion to allow the saliva acquisition device to be inserted into a certain depth, and a light source located in the body and providing light to the saliva acquisition device; and a controller acquiring a captured image of the saliva from the camera portion, extracting a crystal structure image of hormone included in the saliva by processing the acquired captured image, and determining, based on the extracted crystal image structure, whether the subject is in ovulation.

### Advantageous Effects of Disclosure

According to an image processing and analyzing system for ovulation detection and a method of controlling the image processing and analyzing system, a clear captured image of saliva may be acquired by using an ovulation prediction device. In addition, the image processing and analyzing system for ovulation detection may calculate a probability by extracting and classifying crystal structure images from the captured image, and therefore, an ovulation day may be accurately predicted.

### BRIEF DESCRIPTION OF DRAWINGS

FIG. 1 is a conceptual diagram schematically showing an image processing and analyzing system for ovulation detection, according to an embodiment of the present disclosure;

FIG. 2 is a cross-sectional view of an ovulation prediction device shown in FIG. 1, which is taken along line II-II′;

FIG. 3 is a schematic perspective view of a saliva acquisition device shown in FIG. 1;

FIG. 4 is a conceptual diagram for describing an operating method of the saliva acquisition device shown in FIG. 3;

FIG. 5 is an enlarged view showing an S region shown in FIG. 3;

FIG. 6 is a schematic diagram showing correlation between a crystal structure of luteinizing hormone included in saliva and an ovulation cycle;

FIG. 7 is a diagram showing crystal structures of the luteinizing hormone according to an ovulation state;

FIG. 8 is a flowchart sequentially showing a method of controlling an image processing and analyzing system for ovulation detection according to an embodiment of the present disclosure;

FIG. 9 is a conceptual diagram schematically showing an image processing and analyzing system for ovulation detection, according to another embodiment of the present disclosure; and

FIG. 10 is a flowchart showing an order of a method of controlling an image processing and analyzing system shown in FIG. 9.

### BEST MODE

An embodiment of the present disclosure provides an image processing and analyzing system for ovulation detection including: an electronic device including a camera portion; a saliva acquisition device including a saliva accommodation portion accommodating saliva of a subject, and a case in which the saliva accommodation portion is located, the case including an entrance, at an end thereof, through which the saliva accommodation portion enters and exits, and a penetration hole is formed in one surface of the case through which the saliva is be exposed to outside; an ovulation prediction device including a body portion, wherein the electronic device is mounted on one surface and an opening is formed in the surface to allow the camera portion to be positioned in the opening, an insertion unit formed at one side of the body portion to allow the saliva acquisition device to be inserted into a certain depth, and a light source located in the body and providing light to the saliva acquisition device; and a controller acquiring a captured image of the saliva from the camera portion, extracting a crystal structure image of hormone included in the saliva by processing the acquired captured image, and determining, based on the extracted crystal structure image, whether the subject is in ovulation.

In an embodiment, the controller may classify the acquired captured image into a plurality of unit pixels and extract the crystal structure image from the captured image by digitizing each of the plurality of pixels.

In an embodiment, the controller may extract a plurality of corner points from the acquired captured image, extract first pixels that have numerical pixel values within a first numerical value range with reference to a numerical value of the corner points and are continuously connected from the corner point, and determine whether the extracted first pixels correspond to a crystal structure of the hormone based on the extracted first pixels.

In an embodiment, the plurality of corner points may be relatively extracted by comparing numerical pixel values between an arbitrary unit pixel and peripheral pixels neighboring the arbitrary unit pixel in each of a plurality of regions included in the acquired captured image.

In an embodiment, the controller may determine that the first pixels correspond to the crystal structures of the hormones when the first pixels are connected to one another in a length equal to or greater than a first reference value that is set in advance.

In an embodiment, the controller may calculate a length or a thickness of each of the extracted crystal structure images, generate classification data by classifying the extracted crystal structure images based on the length or thickness, and calculate a probability about the ovulation by using the classification data.

In an embodiment, the controller may determine whether the crystal structure image is valid by using directivity of the first pixels.

In an embodiment, an center axis passing through the penetration hole and a center axis passing through the opening may be substantially identical to each other when the saliva acquisition device is inserted into the ovulation prediction device.

In an embodiment, the ovulation prediction device may further include a camera mounting unit, the camera mounting unit including a service entrance that has a shape corresponding to an outer periphery shape of the camera portion and may be attached to and detached from a portion of a surface of the body.

In an embodiment, the ovulation prediction device may further include an optical portion that is located between the opening and the insertion unit and including one or more lens magnifying an image of the saliva.

In an embodiment, the ovulation prediction device may further include a drying unit drying the saliva accommodated in the saliva acquisition device.

In an embodiment, the saliva acquisition device may further include a guiding groove formed in the case in a length direction of the case; and a slider that is connected to a side of the saliva accommodation portion, protrudes to outside of the case, and capable of moving along the guiding groove.

In an embodiment, the saliva acquisition device may further include: a protrusion that is located apart from the entrance in an inner surface of the case and protrudes towards an internal region of the case; and a stopper that is connected to one side of the saliva accommodation portion and holds the saliva accommodation portion by being caught by the protrusion.

In an embodiment, the protrusion may include an incline surface that is inclined by a degree toward the entrance.

In an embodiment, the saliva acquisition device may include a product information portion that is located adjacent to the penetration hole and includes at least one of an encrypted pattern and a serial number.

An embodiment of the present disclosure provides a method of controlling an image processing and analyzing system for ovulation detection including an electronic device which includes a camera portion, a saliva acquisition device in which saliva is accommodated, and an ovulation prediction device into which the saliva acquisition device is inserted, the control method including: inserting the saliva acquisition device, in which the saliva is accommodated, into the ovulation prediction device; acquiring a captured image of the saliva by using the camera portion; extracting a crystal structure image of hormone included in the saliva by analyzing the acquired captured image; and determining whether a subject is in ovulation according to the extracted crystal structure image.

In an embodiment, the extracting of the crystal structure image may include: classifying the acquired captured image into a plurality of unit pixels; and extracting the crystal structure image from the captured image by digitizing each of the plurality of pixel units.

In an embodiment, the extracting of the crystal structure image may include: relatively extracting a plurality of corner points by comparing numerical pixel values between an arbitrary unit pixel and peripheral pixels neighboring the arbitrary unit pixel in each of a plurality of regions included in the acquired captured image; extracting first pixels that have numerical pixel values within a first numerical pixel value range with reference to a numerical pixel value of the corner point and continuously connected from the corner points; and determining, based on the extracted first pixels, whether the extracted first pixels correspond to a crystal structure of the hormone.

In an embodiment, the extracting of the crystal structure image may include determine that the first pixels correspond to the crystal structure of the hormone when the first pixels are connected in a length equal to or greater than a first reference value that is set in advance.

In an embodiment, the determining of whether the subject is in ovulation may include: calculating a length or thickness of each of the extracted crystal structure images; generating classification data by classifying the extracted crystal structure images based on the length or thickness; and calculating a probability about the ovulation by using the classification data.

Aspects and features other than the descriptions will be clearly understood from the accompanying drawings, claims, and the written descriptions.

### MODE OF DISCLOSURE

Hereinafter, following embodiments will now be described more fully with reference to the accompanying drawings, in which like reference numerals denote like or corresponding components in the descriptions with reference to the drawings and repeated descriptions thereof will not be given.

As the present embodiments allow for various changes, particular embodiments will be illustrated in the drawings and described in detail in the written description. Advantages and features of the present embodiments and method of achieving the same may be clear with reference to the attached drawings and following descriptions. However, the present embodiments are not limited to embodiments set forth herein and may be embodied in various forms.

In the following embodiments, terms such as “first,” “second,” and the like are not intended to limit the present disclosure and are intended to distinguish one component from another.

In the following embodiments, an expression used in the singular encompasses the expression of the plural, unless it has a clearly different meaning in the context.

In the following embodiment, terms such as “including,” “comprising,” and “having” are intended to indicate the existence of features or components described in the specification and are not intended to preclude the possibility that one or more other features or components may be added.

It will be understood that when a unit, region, or component is referred to as being “formed on” another unit, region, or component, the unit, region, or component may be directly or indirectly on the other unit, region, or component. That is, for example, intervening units, regions, or components may be present.

In the following embodiments, terms such as “connect,” “combine” do not necessarily indicate that two members are connected or combined directly and/or in a fixed manner, and do not preclude other members may be present between the two members, unless the terms have clearly different meanings in the context.

Terms such as “include,” “comprise,” and “have” are intended to indicate the existence of features or components disclosed in the specification, and are not intended to preclude the possibility that one or more other features or components may be added.

Sizes of components in the drawings may be exaggerated for convenience of explanation. In other words, since sizes and thicknesses of components in the drawings are arbitrarily illustrated for convenience of explanation, the following embodiments are not limited thereto.

[symbol]

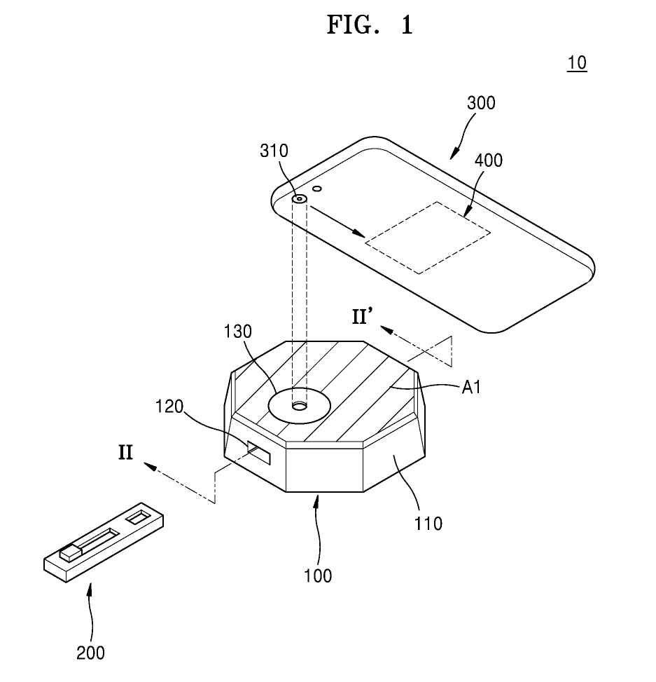

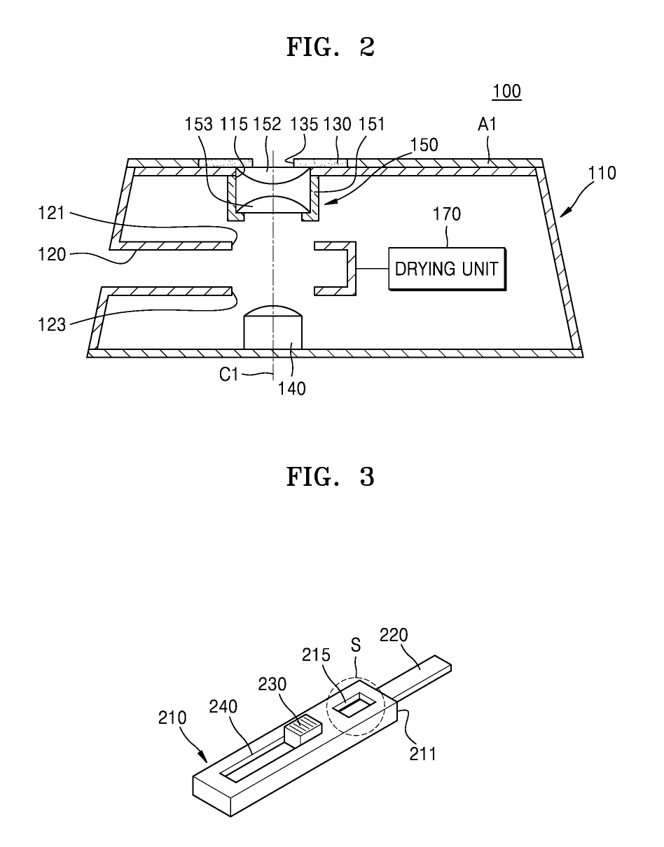

FIG. 1 is a conceptual diagram schematically illustrating an image processing and analyzing system for ovulation detection 10 according to an embodiment of the present disclosure, and FIG. 2 is a cross-sectional view of an ovulation prediction device 100 taken along line II-II′.

Referring to FIG. 1, the image processing and analyzing system for ovulation detection 10 may include the ovulation prediction device 100, a saliva acquisition device 200, an electronic device 300, and a controller 400.

The electronic device 300 that includes a camera portion 310 located in a surface may be a portable device. The electronic device 300 may include a display unit (not shown), the surface may be a surface opposite to a surface in which the display unit (not shown) is located and, as another embodiment, may be the same as the surface in which the display unit (not shown) is located. As another embodiment, the electronic device 300 may include more than two of the camera portions 310 to be located both in the surface at which the display unit (not shown) is located and the surface opposite thereto. For example, the electronic device 300 may be a mobile phone, a tablet PC, a notebook computer, a graphing calculator, a handheld game console, a digital camera, a digital camcorder, a portable media player, and the like.

Although it is not shown, the camera portion 310 may include a lens module including at least one lens and an image sensor sensing an optical image provided to the lens module to take a captured image of the saliva obtaining device 200.

Referring to FIGS. 1 and 2, the ovulation prediction device 100 may include a body portion 110 having the electronic device 300 attached to a surface A1, and an insertion unit 120 formed at one side of the body portion 110 for the saliva acquisition device 200 to be inserted in a predetermined depth. In addition, the ovulation prediction device 100 may include an opening 115 formed at the surface A1 of the body portion 110 unit to accommodate the camera portion 310 of the electronic device 300, and a light source 140 that is located in the body portion 110 and emits light to the saliva acquisition device 200.

The body portion 110 of the ovulation prediction device 100 may be formed in a certain size and shape. The body portion 110 may include the surface A1 that is an upper surface, which is an upper surface located in a direction perpendicular to a direction in which the saliva acquisition device 200 is inserted through the insertion unit 120, and the electronic device 300 may be mounted on the surface A1. The surface A1 of the body portion 110 may include a material which may apply moderated frictional force to the electronic device 300 such that the electronic device 300 is stably mounted on the body portion 120. For example, the surface A1 of the body portion 110 may include rubber. However, the present disclosure is not limited thereto and various materials may be used for stably mounting the electronic device 300. Meanwhile, the opening 115 may be formed in the surface A1 of the body portion 110 such that the camera portion 310 of the electronic device 300 may be accommodated. The camera portion 310 of the electronic device 300 mounted in the body portion 110 may face the saliva acquisition device 200 through the opening 115. Meanwhile, when the saliva acquisition device 200 is inserted into the ovulation prediction device 100, the opening 115 may be formed at a position where a central axis passing through a penetration hole 215 and a central axis passing through the opening 115 meets each other.

The insertion unit 120 may be formed at a side of the body portion 110 such that the saliva acquisition device 200 is inserted in a predetermined depth. As the insertion unit 120 is formed in a size and shape corresponding to that of the saliva acquisition device 200, the saliva acquisition device 200 may be stably inserted into the insertion unit 120. In the insertion unit 120, a first exposure unit 121 may be formed at a position to overlap the opening 115 such that the camera portion 310 of the electronic device 300 may shot the saliva acquisition device 200 that is inserted. In addition, a second insertion unit 123 may be formed such that light of the light source 140 may be emitted to the saliva acquisition device 200. The first exposure unit 121 and the second exposure unit 123 may be on a same straight line with the opening 115 of the body portion 110. Sizes of the first exposure unit 121 and the second exposure unit 123 may be different from each other. For example, the size of the second exposure unit 123 may be greater than the size of the second exposure unit 121. The light emitted from the light source 140 is incident on the second exposure unit 123, and thus, the size of the second exposure unit 123 may be greater than the size of the first exposure 121.

The light source 140 may be located in the body portion 110 and provide light to the saliva acquisition device 200. The light source 140 may employ all types of source devices capable of providing light to the saliva acquisition device 200. For example, the light source 140 may be a light-emitting device (LED). In an embodiment, as shown in FIG. 2, the light source 140 may be on an extension line C1 passing through a center of the opening 115 in the body portion 110. However, the present embodiment is not limited thereto and the light source 140 may be located at any position that enables the camera portion 310 of the electronic device 300 to shot the saliva acquisition device 200 by using the light provided from the light source 140. For example, when the light source 140 is not on the extension line C1 passing through the center of the opening 115, the ovulation prediction device 100 may further include a light path changing unit (not shown) changing a light path such that the light provided from the light source 140 is transmitted to the saliva acquisition device 200. The light path changing unit may include a mirror or a lens and guide the light from the light source 140 to the saliva acquisition device 200.

In addition, only one light source 140 is shown in the drawings, the ovulation prediction device 100 may include more than one light source 140. The ovulation prediction device 100 may provide light to the saliva acquisition device 200 from the light source 140 and to an external area of the ovulation prediction device 100. In this case, the body portion 110 includes a transparent or translucent material and the light emitted from the light source 140 may be transmitted to the external region of the body portion 110. By doing so, the ovulation prediction device 100 may also function as a flashlight. When functioning as a flashlight, the ovulation prediction device 100 may be used in a reversed state such that the surface A1 of the body portion 110 is a lower surface.

Meanwhile, the ovulation prediction device 100 may further include an optical portion 150, a drying unit 170, a camera mounting unit 130, and a power unit (not shown).

The optical portion 150 may be located between the opening 115 and the insertion unit 120 of the body portion 110 and include more than one lens for magnifying a saliva image. In an embodiment, the optical portion 150 may include a wide angle lens 152, a macroscopic lens 153, and a fixing member 151 for fixing the wide angle lens 152 and the macroscopic lens 153. The optical portion 150 may use the wide angle lens 152 and the macroscopic lens 153 to magnify the saliva image of the saliva acquisition device 200 inserted into the body portion 110 and may also be fixed in the body portion 110 to maintain a certain focus distance. The optical portion 150 is fixed in the body portion 110, and a distance between the camera portion 310 of the electronic device 300 and the optical portion 150 and a distance between the optical portion 150 and the saliva acquisition device 200 may be constant. Accordingly, the image processing and analyzing system for ovulation detection 10 may acquire a clear captured image of the saliva by using the ovulation prediction device 100.

The drying unit 170 may dry the saliva accommodated in the saliva acquisition device 200. The drying unit 170 may use at least one of air and heat. For example, the drying unit 170 may include a fan to dry the saliva accommodated in the saliva acquisition device 200. As another embodiment, the drying unit 170 may use heat provided from a Peltier device or electrical resistance heat to dry the saliva accommodated in the saliva acquisition device 200. Alternatively, the drying unit 170 may include both the fan and the Peltier device and dry the saliva by using air and heat. Meanwhile, the drying unit 170 may be integrally formed with the light source 140. More particularly, the light source 140 may include an incandescent lamp and use heat generated by driving the incandescent lamp to dry the saliva. Drying devices applying various drying methods may be further used.

The camera mounting unit 130 includes a service entrance 135 having a shape corresponding to a shape of an outer periphery shape of the camera portion 30 and may be attached to and detached from a portion of the surface A1 of the body portion 110. When the electronic device 300 is a smart phone on the market, the shape of the camera portion 310 of the smart phone may depend on the types of the smart phone. The camera mounting unit 130 may correspond to the outer shape of the camera portion 310 of the user and enables the camera portion 310 to stably acquire a captured image of the saliva. In this case, a central axis of the service entrance 135 may be equal to the central axis of the opening 115. The camera mounting unit 130 may be formed in a disposable manner and located in the surface A1 of the body portion 110 as needed. Meanwhile, the surface A1 of the body portion 110 may further include a mounting groove (not shown) having a shape corresponding to an outer periphery shape of the camera mounting unit 130, and the electronic device 300 may be stably mounted on the body portion 110 through the mounting groove.

Meanwhile, the ovulation prediction device 100 may further include a power unit (not shown) providing electric power to the light source 140 or the drying unit 170. The power unit may be connected to an external power source or provide power itself by using a built-in battery. When providing power by using the built-in battery, the ovulation prediction device 100 may perform a function of a portable power bank by using the built-in battery. Although it is not shown, in this case, the ovulation prediction device 100 may further include a charging port charging the built-in battery and a providing port providing power of the battery to an external area.

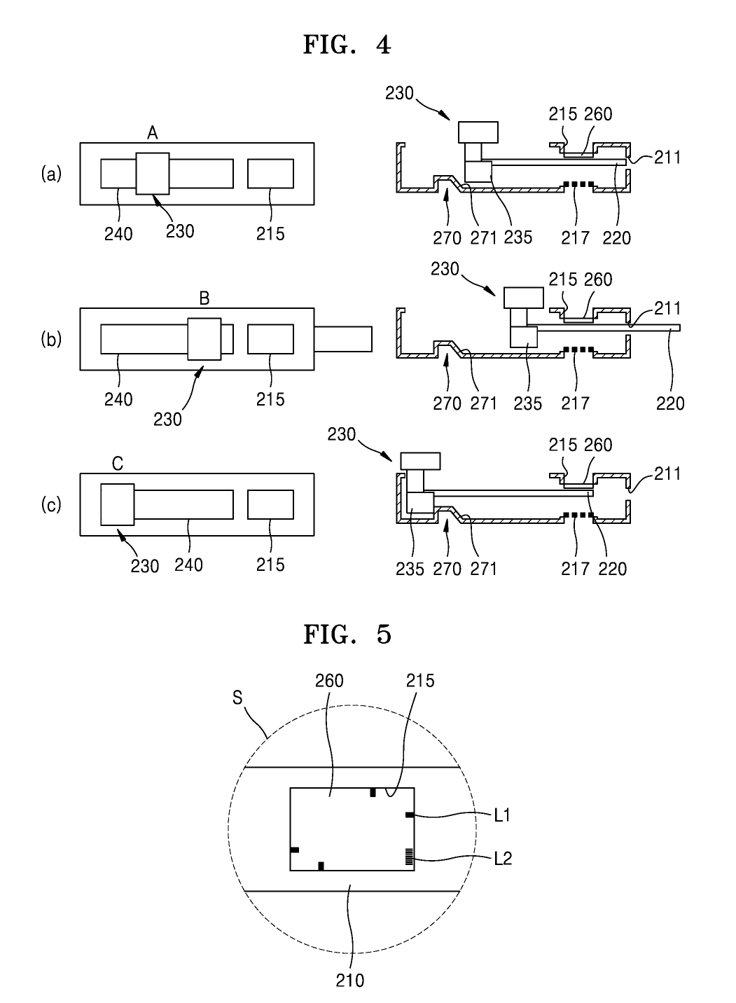

FIG. 3 is a schematic perspective view showing the saliva acquisition device 200 of FIG. 1, and FIG. 4 is a conceptual diagram for describing an operating method of the saliva acquisition device 200 of FIG. 3. FIG. 5 is an enlarged view showing an S region shown in FIG. 3.

Referring to FIGS. 3 and 4, the saliva acquisition device 200 may include a case 210, a saliva accommodation portion 220, a slider 230, and a guiding groove 240.

The saliva accommodation portion 200 may accommodate saliva of a subject. Here, the saliva may indicate saliva including luteinizing hormone that may be used for predicting ovulation. The saliva accommodation portion 220 may include a transparent material such as acryl. In addition, the saliva accommodation portion 220 may include a flexible material such that the saliva may be easily acquired from an upper portion and a lower portion of a tongue of the subject. Furthermore, the saliva accommodation portion 220 may protrude toward outside of the case 210 such that the user may easily acquire the saliva.

The saliva accommodation portion 220 may be located in the case 210. An entrance 211 through which the saliva accommodation portion 200 may enter or exit may be formed at an end of the case 210, and the penetration hole 215 may be formed at a surface of the case 210 such that the saliva is exposed to the external region of the case 210. The guiding groove 240 may be formed in the case 210 in a length direction of the case 210. Although FIG. 3 shows an embodiment in which the guiding groove 240 and the penetration hole 215 are formed in a same surface, the present disclosure is not limited thereto. The guiding groove 240 may be formed in another surface other than the surface in which the penetration hole 215 is formed. Meanwhile, a vent 217 penetrating another surface opposite to the penetration hole 215 may be further formed in the case 210. In the saliva acquisition device 200, air or heat provided from the drying unit 170 is delivered to the saliva through the vent 217, and the saliva may be effectively dried. As another embodiment, the vent 217 may be in another surface of the case 210 other than the other surface opposite to the penetration hole 215.

The slider 230 may be connected to one side of the saliva accommodation portion 220, protrude to outside of the case 210, and move along the guiding groove 240. In other words, the user may transfer the saliva accommodation portion 220 to the external region or an internal region of the case 210 by pushing or pulling the slider 230. The slider 230 may include a frictional material or have a plurality of protrusions on an upper surface such that the user may easily operate the slider 230.

Referring (a) of FIG. 4, the slider 230 of the saliva acquisition device 200 may be at ‘A’ before a product is used. In this case, the saliva accommodation portion 220 may be located in the case 210 to be protected from contamination. To take the saliva by using the saliva accommodation portion 220, the slider 230 is moved to ‘B’ along the guiding groove 240 as shown in (b) of FIG. 4 such that the saliva accommodation portion 220 is exposed to outside of the saliva acquisition device 200 through the entrance 211. The user may acquire the saliva of the subject by using the saliva accommodation portion 220 that is exposed to the external region of the saliva acquisition device 200.

Referring to (c) of FIG. 4, the user may bring the saliva accommodation portion 220 into the case 210 by moving the slider 230 to ‘C’. The saliva accommodated in the saliva accommodation portion 220 may be exposed to outside through the penetration hole 215. In this state, the saliva acquisition device 200 may be inserted into the ovulation prediction device 100 to acquire a captured image of the saliva by using the camera portion 310 of the electronic device 300.

Meanwhile, the saliva acquisition device 200 may be used in a disposable manner for sanitation and accurate analysis. The saliva acquisition device 200 may further include a protrusion 270 and a stopper 235 for disposability.

The protrusion 270 may be located apart from the entrance 211 in an inner side of the case 210 and may protrude toward outside of the case 210. The stopper 235 may be connected to a side of the saliva accommodation portion 220 and fix the saliva accommodation portion 220 by being caught by the protrusion 270. In this case, the protrusion 270 may include an incline surface 271 inclined in a predetermined degree toward the entrance 211 to enable the stopper 235 to easily enter the case 235. By doing so, the stopper 235 and the protrusion 270 are unidirectional and may not be reused once the stopper 235 passes the protrusion 270. Meanwhile, the stopper 235 may be integrally formed with the slider 230.

Referring to FIG. 5, the saliva acquisition device 200 may further include a product information portion 260. The product information portion 260 is located adjacent to the penetration hole 215 and may include at least one of an encrypted pattern and a serial number. The encrypted pattern may include a bar code L2, a Quick Response (QC) code, a character code, a graphic code L1, and the like. Alternatively, the encrypted pattern may include an edge structure of the product information portion that is formed into a pattern. The saliva acquisition device 200 may confirm whether the product is genuine or at a normal position by using the product information portion 260. In other words, when the saliva acquisition device 200 is inserted into the ovulation prediction device 100, not only the captured image of the saliva but also the image of the product information portion 260 may be acquired. By doing so, it may be determined whether saliva acquisition device 200 is a normal product of the company, or it may be identified whether the saliva acquisition device 200 is inserted into a normal position and notify results of the determination and identification to the user. The product information portion 260 may locate the encrypted pattern or the serial number on a surface of the case 210 neighboring the penetration hole 215, however, in an embodiment, the product information portion 260 may include a transparent window located in the penetration hole 215 and insert the encrypted pattern or the serial number into the transparent window. Meanwhile, the product information portion 260 may include a chip such as a radio frequency identification (RFID) chip or an integrated circuit (IC) chip. The image processing and analyzing system for ovulation detection 10 according to an embodiment may recognize the encrypted pattern by using a captured image of the product information portion 260 or may further include a pattern recognizing unit (not shown) for recognizing the encrypted pattern. For example, the image processing and analyzing system for ovulation detection 10 may further include a component linked to a reader such as RFID or IC.

Meanwhile, the controller 400 may acquire a captured image of saliva by using the camera portion 310 and extract a crystal structure image of hormone included in the saliva by processing the captured image of saliva. In addition, the controller 400 may determine whether the subject is with ovulation based on the extracted crystal structure image. Here, the controller 400 may include all types of devices capable of processing data, for example, a processor. The term ‘processor’ may indicate a data processing device mounted in hardware, wherein the processor has a physically structurized circuit for executing functions expressed as codes or commands included in a program. The data processing device embedded in hardware may include processing devices such as a microprocessor, a central processing unit (CPU), a processor core, a multiprocessor, an application-specific integrated circuit (ASIC), a field programmable gate array (FPGA), but the scope of the present disclosure is not limited thereto.

In an embodiment, the controller 400 may be located in the electronic device 300. The controller 400 may implement the above-mentioned operations by driving an application program or an application stored in a memory (not shown) of the electronic device 300. As another embodiment, the controller 400 may be located in the ovulation prediction device 100. In this case, the ovulation prediction device 100 may independently include a camera portion to process and analyze an image without using the electronic device 300. However, for convenience of explanation, an embodiment in which the controller 400 is located in the electronic device 300 is mainly described hereinafter.

Hereinafter, a method of controlling the image processing and analyzing system for ovulation detection 10 according to an embodiment of the disclosure, by the controller 400, is described.

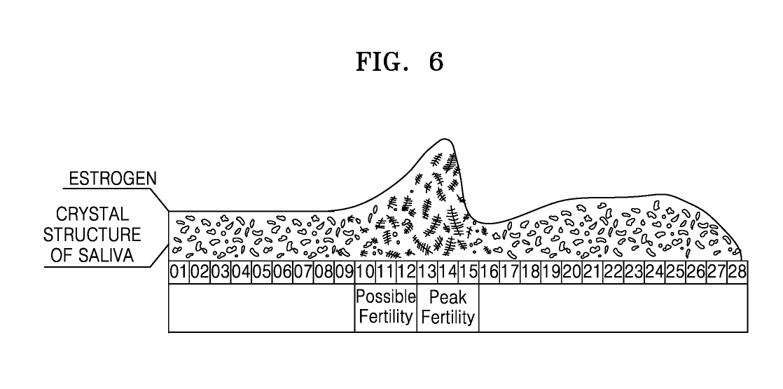

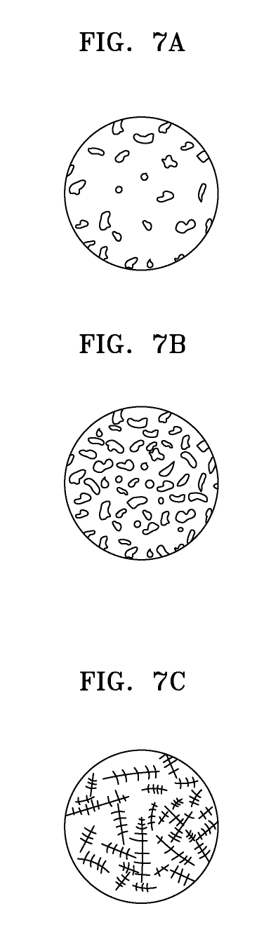

FIG. 6 is a schematic diagram of a correlation between a crystal structure of luteinizing hormone (LH) included in the saliva and an ovulation cycle, and FIG. 7 is a diagram showing crystal structures of LH according to an ovulation state.

Referring to FIG. 7, it is understood that a crystal structure of LH included in the saliva changes according to the ovulation cycle. The change in the crystal structure may be used for identifying an infertility period ((a) of FIG. 7), a transition period ((b) of FIG. 7) and an ovulation period ((c)) of FIG. 7). LH included in the saliva may, in the ovulation period, have a fern-type crystal structure in which straight lines cross one another. However, it is difficult to accurately identify accurate ovulation by using human eyes even when the saliva includes the fern-type crystal structure. The image processing and analyzing system for ovulation detection according to the embodiment 10 may extract the crystal structure image from the captured image of saliva acquired from the camera portion 310 and provide an accurate probability about ovulation by analyzing the crystal structure images.

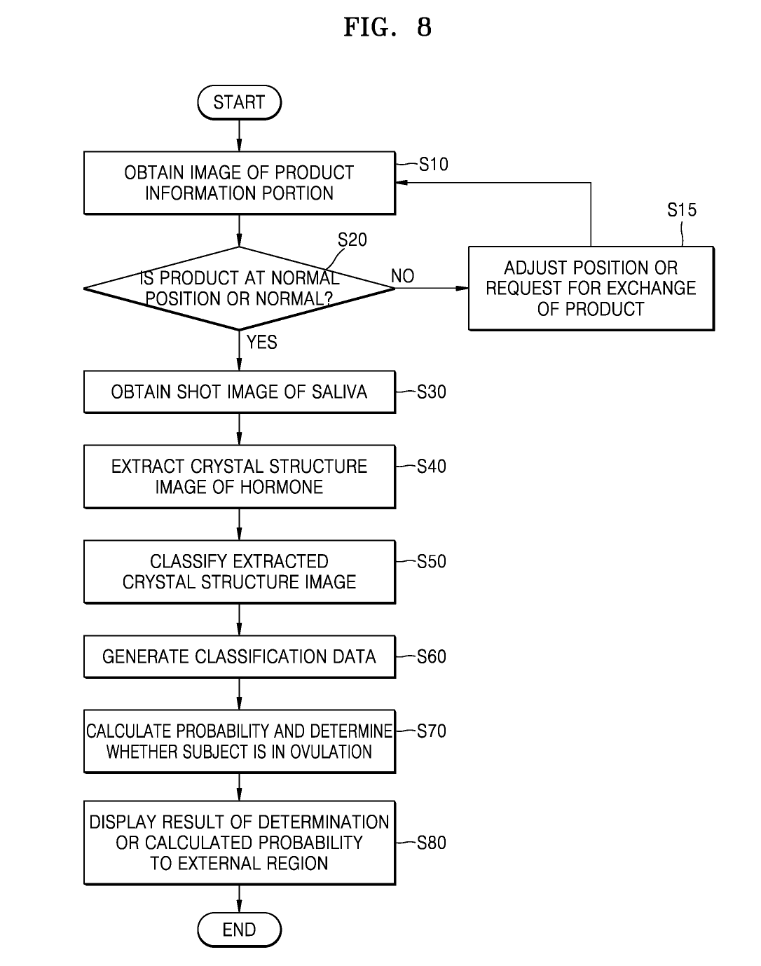

FIG. 8 is a flowchart sequentially showing a method of controlling the image processing and analyzing system for ovulation detection 10 according to an embodiment of the present disclosure, in the controller 400.

Referring to FIG. 8, personal information and hormone information including menstrual cycle information of the subject may be stored in advance in the controller 400. When an application is executed by the electronic device 300, a main page including an initial standby screen, a calendar screen, or setting may be activated. In this case, personal information of the user may be stored in the application to store periodical or non-periodical data of the user. The personal information or the hormone information may be directly input and stored after the user drives the application, or alternatively, determination information derived whenever the ovulation is detected may be stored in database (not shown) of the controller 400. Personal information of the subject may include a height, a weight, a date of birth, a last menstrual date for last three months, days of menstrual cycle, and the like.

Next, when the saliva acquisition device 200 is inserted into the ovulation prediction device, the controller 400 may first obtain an image of the product information portion 260 of the saliva acquisition device 200 (S10). As the product information portion 260 includes at least one of the encrypted pattern and the serial number, the controller 400 may identify whether the product is genuine or at a normal position from the image of the product information portion 260 (S20). Alternatively, the controller 400 may identify the number of use, a coupling state, and experience of cleaning by using the encrypted pattern or the serial number. Alternatively, the controller 400 may identify whether the product is genuine by analyzing a color of the saliva accommodation portion 220. In this case, when the saliva acquisition device 200 that is inserted is not genuine nor a normal product, or when the saliva acquisition device 200 is at a wrong position, the controller 400 may send a request to the user to replacement of the product or adjustment the position of the saliva acquisition device 200 that is located at the wrong position (S15).

When the saliva acquisition device 200 that is inserted is genuine and placed at a normal position, the controller 400 may acquire the captured image of the saliva by using the camera portion 310 (S30) and may extract the crystal structure image of the hormone included in the saliva (S40) by processing the acquired captured image. The captured image of the saliva may include not only the crystal structure but also a foreign material. To distinguish the crystal structure of the hormone from the foreign material, the controller 400 may classify the captured image into a plurality of unit pixels and quantify each of the plurality of unit pixels to extract a crystal structure image from the captured image. For example, each value of the plurality of unit pixels may be classified into a gray scale ranging from level 0 to level 255. In this case, the controller 400 may relatively extract a plurality of corner points from each of a plurality of regions included in the captured image by comparing quantities of an arbitrary unit pixel and peripheral pixels neighboring the arbitrary unit pixel. Here, a corner point may be a start point or an ending point in a length direction of the crystal structure of the hormone Particularly, when the corner point exists in a region, peripheral pixels neighboring the corner point may have a numerical pixel value greater than that of the corner point. In other words, when peripheral pixels neighboring an arbitrary unit pixel have a numerical pixel value greater than that of the arbitrary unit pixel and are connected to one another along a periphery, the arbitrary unit pixel may be extracted as the corner point.

Next, the controller 400 may extract first pixels that have numerical pixel values within a first numerical pixel value range with reference to the numerical pixel value of the corner point and connected one another starting from the corner points. Here, the first pixels may be unit pixels having a value similar to the numerical pixel values of the corner points. When the first pixels are continuously connected to one another starting from the corner point, the controller 400 may determine that the first pixels that are extracted include a fern-type structure in which lines are connected to one another, as shown in (c) of FIG. 7.

When the first pixels are connected in a length equal to or greater than a first reference value that is set in advance, the controller 400 may determine that the first pixels have crystal structure of the hormone. In other words, even when the first pixels are consequently connected to one another starting from the corner point, when a connected length of the first pixels is less than or equal to the first reference value, the controller 400 may determine that the first pixels do not have the fern-type crystal structure corresponding to the ovulation and may not extract the crystal structure image. The first reference value, which is a reference of the length, may vary according to subjects, and may be determined based on personal information and hormone information of the subject. In addition, the controller 400 may determine whether the crystal structure image is valid by using a directivity of the first pixels. For example, the luteinizing having the fern-type structure has a long shape instead of a circle, and when the extracted pixels are unidirectional, the crystal structure image may be determined as valid.

Next, the controller 400 may calculate a length or thickness of each of the extracted crystal structure images and may classify the extracted crystal structure images based on lengths or thicknesses (S50). The controller 400 may generate classification data S60 by matching classification items and the number of crystal structures images corresponding to each of the classification items. Referring to (c) of FIG. 7, a plurality of luteizing hormones may form a plurality of crystal structures in the saliva, and the plurality of crystal structures each have a length and thickness. The controller 400 may generate classification data by classifying valid crystal structure images according to lengths or thicknesses. Next, the controller 400 may calculate, by using the classification data, a probability about whether the subject is in ovulation (S70). For example, grades may be given to the classification items of the extracted crystal structure images, the number of crystal structure images corresponding to each grade may be digitized, and thus, the probability about occurrence of ovulation may be calculated. In an embodiment, when fifteen to twenty of the first pixels are continuously connected to one another, the controller 400 may classify a crystal structure image including the first pixels as a first class, and when ten to fifteen of the first pixels are continuously connected to one another, the controller 400 may classify a crystal structure image including the first pixels as a second class. The controller 400 may score the first grade as 90 points and score the second grade as 70 points. The controller 400 may calculate a total point for the entire captured image by using the point of the grades and the number of crystal structure images included in each grade, and may calculate the probability about occurrence of ovulation by using the total point. However, it is merely an example and the present disclosure is not limited thereto.

The controller 400 may display the calculated probability to the external region or may determine ovulation and display the ovulation to the external region (S80). The user may choose displaying the calculated probability or displaying a result of determining of the ovulation. The controller 400 may determine that the subject is in ovulation when the probability about the ovulation is equal to or greater than the reference probability that is set in advance. In this case, the reference probability may be determined based on existing data base of the subject or may be determined by an arbitrary choice of the user.

As described above, according to the image processing and analyzing system for ovulation detection 10 and a method of controlling the image processing and analyzing system, a clear captured image of saliva may be acquired by using the ovulation prediction device. In addition, the image processing and analyzing system for ovulation detection may calculate a probability by extracting and classifying crystal structure images from the captured image, and therefore, ovulation may be accurately predicted.

[symbol]

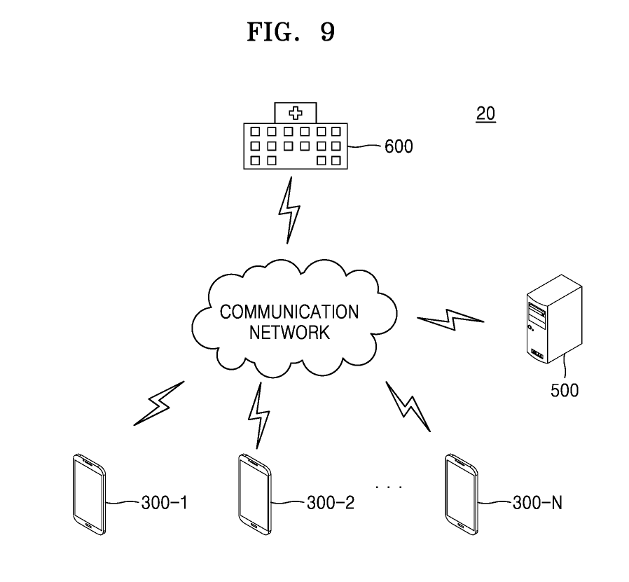

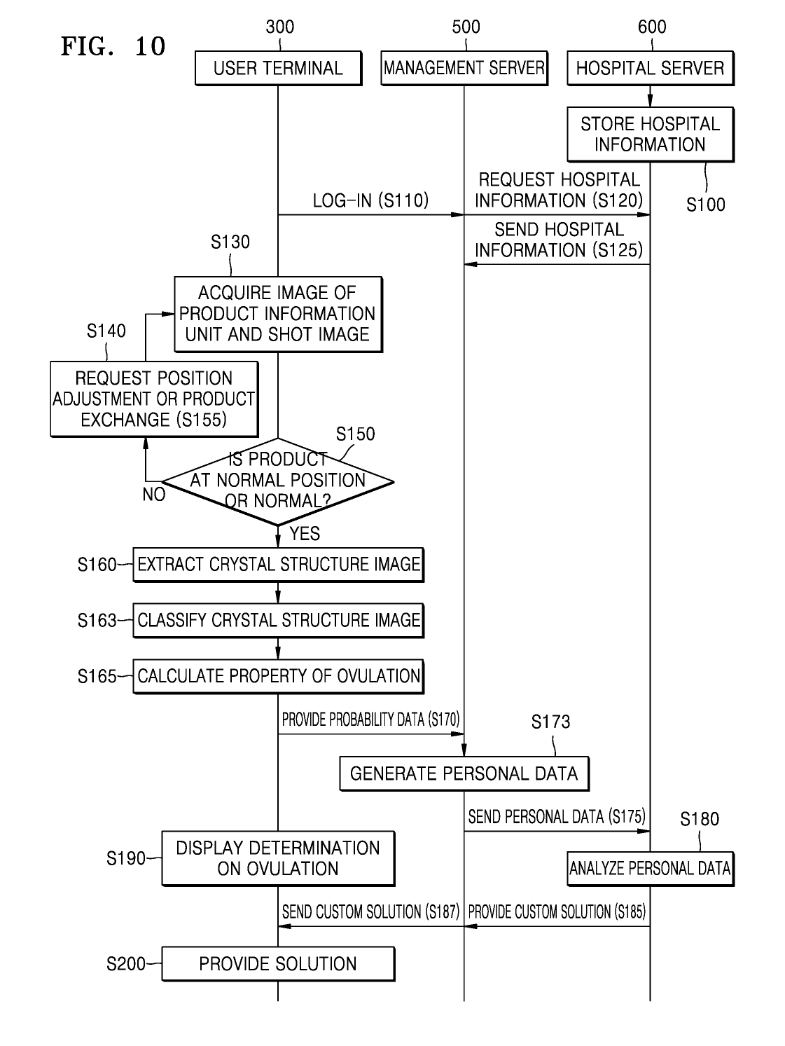

FIG. 9 is a conceptual diagram schematically showing an image processing and analyzing system for ovulation detection 20 according to another embodiment of the present disclosure and FIG. 10 is a flowchart showing an order of method of controlling the image processing and analyzing system ovulation detection 20 shown in FIG. 9.

Referring to FIGS. 9 and 10, the image processing and analyzing system for ovulation detection 20 according to the other embodiment may include an ovulation prediction device (not shown), a saliva acquisition device (not shown), the plurality of electronic devices 300, a management server 500, and a hospital server 600. The image processing and analyzing system for ovulation detection 20 according to the other embodiment includes same components as the ovulation prediction device 100, the saliva acquisition device 200, and the electronic device 300 in an embodiment and may further include the management server 500 and the hospital server 600. For convenience of explanation, repeated description will be omitted. In this case, a controller may be in each of the plurality of electronic device 300.

The electronic device 300 may include the camera portion 310. The user may take saliva of the subject by using the saliva acquisition device 200, insert the saliva into the ovulation prediction device 100, and generate a captured image of the saliva by using the camera portion 310.

The hospital server 600 may store hospital information of the subject (S100) identified from the hospital, for example, ovulation information, hormone information, and the like.

The management server 500 may store personal information of a plurality of subjects, and the personal information of the plurality of subjects may include a height, a weight, a date of birth, a last menstrual date for the last three months, days in a menstrual cycle, and the like.

First, an application which may be connected to the management server may be installed in each of the plurality of electronic device 300 and the user may log-in by driving the application (S110). When a log-in signal is input, the management server 500 may, to the hospital server 600, send a request for the hospital information of the subject (S120). It is shown as the user sends the request for the hospital information after a log-in request and the hospital server 600 transmits the hospital information (S125). However, the present disclosure is not limited thereto, and the hospital server 600 may send the hospital information to the management server 500 in advance such that the information is stored in advance.

The user may shot the saliva in the saliva acquisition device 200 by using the electronic device 300. In this case, the camera portion 310 of the electronic device 300 may simultaneously shot an image of the product information portion of the saliva acquisition device 200 and acquire a p image and a captured image (S130).

The electronic device 300 may first determine whether the saliva acquisition device 200 is a genuine or normal product or determine whether the saliva acquisition device 200 is normally inserted by using an encrypted pattern or a serial number in the product information image (S150). When the saliva acquisition device 200 is a normal product and is at a normal position, the electronic device 300 may extract a crystal structure image by using the captured image (S160). However, when the saliva acquisition device 200 is not at a normal position, or when the saliva acquisition device 200 is not a genuine or normal product, the electronic device 300 may send a message instructing the user to adjust the position or change the product (S140).

When the saliva acquisition device 200 that is inserted is genuine and is at a normal position, the electronic device 300 may extract a crystal structure image of the hormone included in the saliva (S160) by processing a captured image of the saliva. The electronic device 300 may classify the acquired captured images into a plurality of unit pixels, digitize each of the plurality of unit pixels, and extract a crystal structure image from the captured images. A corner point may be extracted in the above-mentioned process, and first pixels that have numerical pixel values within the first numerical pixel value range with reference to the numerical pixel value of the corner point and connected to one another starting from the corner point. The electronic device 300 may extract the crystal structure image by using the extracted first pixels.

Next, the electronic device 300 may calculate a length or thickness of each of the extracted crystal structure images and classify the extracted crystal structure images based on lengths and thicknesses (S163). The electronic device 300 may generate classification data by matching classification items and the number of crystal structure images corresponding to each of the classification items. The classification data may be generated by classifying valid crystal structure images corresponding to a reference level by using lengths and thicknesses.

The electronic device 300 may calculate, by using the classification data, a probability about whether the subject is in ovulation (S165). The electronic device 300 may display the calculated probability to the external region or may determine ovulation and display the ovulation to the external region (S190). The user may choose displaying the possibility or displaying a result of determining of the ovulation. The electronic device 300 may determine that the subject is in ovulation when the probability about the ovulation is equal to or greater than a reference probability that is set in advance. In this case, the reference probability may be determined based on existing data base of the subject or may be determined by an arbitrary choice of the user. The electronic device 300 may include a communication unit (not shown) that may with the management server 500 and may transmit, to the management server 500, probability data including a probability about whether the subject is in ovulation, which is generated through wired or wireless communication networks.

The management server 500 may generate personal data by linking the transmitted probability data and personal information. Personal data regarding a plurality of subjects may be stored in the management server 500. That is, big data may be stored in the management server 500. In addition, the management server 500 may transmit the personal data to the hospital server 600. In this case, the hospital server 600 may analyze the personal data by using the big data stored in the management server 500 (S180). For example, the management server 500 may include big data, in which personal information including body mass indices (BMI), menstrual cycles, weights, heights, amounts of exercise of a plurality of subjects is linked to the probability data received from the electronic device 300. The hospital server 600 may, based on the big data, compare and analyze a subject who needs a customized solution and a comparative subject having a weight and a height similar to those of the subject, and may provide a customized solution for increasing the amount of exercise or lowering a BMI index to improve the probability of ovulation. The hospital server 600 may send the customized solution directly to the electronic device 300, may provide the customized solution to the management server (S185), and may have the customized solution to be sent to the electronic device 300 through the management server (S187). The electronic device 300 may display the customized solution to the external region (S200).

The embodiments have been described with reference to example embodiments. It will be understood by those of ordinary skill in the art that various changes in forms and details may be made therein without departing from the spirit and scope as defined by the following claims. Therefore, the disclosed embodiments are to be considered in a descriptive sense only and not for purposes of limitation. Therefore, the scope of the present disclosure is defined not by the detailed description but by the appended claims, and all differences within the scope will be construed as being included in the present disclosure.

### INDUSTRIAL APPLICABILITY

According to an embodiment, an image processing and analyzing system for ovulation detection is provided. In addition, the embodiments may be applied to a hormone analyzing tool and the like that is used in industrial purposes.

1. An image processing and analyzing system for ovulation detection, the image processing and analyzing system comprising:
an electronic device comprising a camera portion;
a saliva acquisition device comprising a saliva accommodation portion accommodating saliva of a subject, and a case in which the saliva accommodation portion is located, the case comprising an entrance, at an end thereof, through which the saliva accommodation portion enters and exits, and a penetration hole formed in one surface of the case through which the saliva is exposed to outside;
an ovulation prediction device comprising a body portion, wherein the electronic device is mounted on one surface and an opening is formed in the surface to allow the camera portion to be positioned in the opening, an insertion unit formed at one side of the body portion to allow the saliva acquisition device to be inserted in a certain depth, and a light source that is located in the body portion and provides light to the saliva acquisition device; and
a controller acquiring a captured image of the saliva from the camera portion, extracting a crystal structure image of hormone included in the saliva by processing the acquired captured image, and determining, based on the extracted crystal structure image, whether the subject is in ovulation.

2. The image processing and analyzing system of claim 1, wherein the controller classifies the acquired captured image into a plurality of unit pixels and extracts the crystal structure image from the captured image by digitizing each of the plurality of unit pixels.

3. The image processing and analyzing system of claim 2, wherein the controller extracts a plurality of corner points from the acquired captured image, extracts first pixels that have numerical pixel values within a first numerical pixel value range with reference to numerical pixel values of the corner points and are continuously connected from the corner points, and determines whether the extracted first pixels correspond to a crystal structure of the hormone based on the extracted first pixels.

4. The image processing and analyzing system of claim 3, wherein the plurality of corner points are relatively extracted by comparing numerical pixel values between an arbitrary unit pixel and peripheral pixels neighboring the arbitrary unit pixel in each of a plurality of regions included in the acquired captured image.

5. The image processing and analyzing system of claim 3, wherein the controller determines the first pixels correspond to the crystal structure of the hormones when the first pixels are connected in a length equal to or greater than a first reference value that is set in advance.

6. The image processing and analyzing system of claim 3, wherein the controller calculates a length or a thickness of each of the extracted crystal structure images, generates classification data by classifying the extracted crystal structure images based on the length or thickness, and calculate a probability about ovulation by using the classification data.

7. The image processing and analyzing system of claim 6, wherein the controller determines whether the crystal structure image is valid by using directivity of the first pixels.

8. The image processing and analyzing system of claim 1, wherein a center axis passing through the penetration hole and a center axis passing through the opening are substantially identical to each other when the saliva acquisition device is inserted into the ovulation prediction device.

9. The image processing and analyzing system of claim 1, wherein the ovulation prediction device further comprises a camera mounting unit, the camera mounting unit including a service entrance that has a shape corresponding to an outer periphery shape of the camera portion and is attached to and detached from a portion of a surface of the body portion.

10. The image processing and analyzing system of claim 1, wherein the ovulation prediction device further comprises an optical portion that is located between the opening and the insertion unit and includes one or more lenses magnifying an image of the saliva.

11. The image processing and analyzing system of claim 1, wherein the ovulation prediction device further comprises a drying unit drying the saliva accommodated in the saliva acquisition device.

12. The image processing and analyzing system of claim 1, wherein the saliva acquisition device further comprises: a guiding groove formed in the case in a length direction of the case; and a slider that is connected to a side of the saliva accommodation portion, protrudes to outside of the case, and capable of moving along the guiding groove.

13. The image processing and analyzing system of claim 12, wherein the saliva acquisition device further comprises a protrusion that is located apart from the entrance in an inner surface of the case and protrudes toward an internal region of the case; and a stopper that is connected to one side of the saliva accommodation portion and holds the saliva accommodation portion by being caught by the protrusion.

14. The image processing and analyzing system of claim 13, wherein the protrusion comprises an incline surface that is inclined by a degree toward the entrance.

15. The image processing and analyzing system of claim 1, wherein the saliva acquisition device further comprises a product information portion that is located adjacent to the penetration hole and comprises at least one of an encrypted pattern and a serial number.

16. A method of controlling an image processing and analyzing system for ovulation detection comprising an electronic device which comprises a camera portion, a saliva acquisition device in which saliva is accommodated, and an ovulation prediction device into which the saliva acquisition device is inserted, the method comprising: inserting the saliva acquisition device, in which the saliva is accommodated, into the ovulation prediction device; acquiring a captured image of the saliva by using the camera portion; extracting a crystal structure image of hormone included in the saliva by analyzing the acquired captured image; and determining whether a subject is in ovulation according to the extracted crystal structure image.

17. The method of claim 16, wherein the extracting of the crystal structure image comprises; classifying the acquired captured image into a plurality of unit pixels; and extracting the crystal structure image from the captured image by digitizing each of the plurality of unit pixels.

18. The method of claim 17, wherein the extracting of the crystal structure image comprises: relatively extracting a plurality of corner points by comparing numerical pixel values between an arbitrary unit pixel and peripheral pixels neighboring the arbitrary unit pixel in each of a plurality of regions included in the acquired captured image; extracting first pixels that have numerical pixel values within a first numerical value range with reference to numerical pixel values of the corner points and continuously connected from the corner points; and determining, based on the extracted first pixels, whether the extracted first pixels correspond to a crystal structure of the hormone.

19. The method of claim 18, wherein the extracting of the crystal structure image comprises determining the first pixels correspond to the crystal structure of the hormone when the first pixels are connected to one another in a length equal to or greater than a first reference value that is set in advance.

20. The method of claim 19, wherein the determining of whether the subject is in ovulation comprises: calculating a length or thickness of each of the extracted crystal structure images; generating classification data by classifying the extracted crystal structure images based on the length or the thickness; and calculating a probability about ovulation by using the classification data.
